(19) United States
(12) Patent Application Publication
(10) Pub. No.: US 2019/0093468 A1
(43) Pub. Date: Mar. 28, 2019

(54) REAL TIME MEASUREMENT OF MUD PROPERTIES FOR OPTIMIZATION OF DRILLING PARAMETERS

(71) Applicant: Schlumberger Technology Corporation, Sugar Land, TX (US)

(72) Inventors: Franz Aguirre, Sugar Land, TX (US); Andrew Marlatt, Houston, TX (US)

(21) Appl. No.: 15/717,659

(22) Filed: Sep. 27, 2017

(51) Int. Cl. E21B 44/00; G01N 33/28; E21B 21/00; E21B 21/06; G06Q 30/02

(52) U.S. Cl. CPC E21B 44/00; G01N 33/2823; E21B 47/18; E21B 21/065; G06Q 30/0206; E21B 21/00

(57) ABSTRACT

A system includes a continuous mud measurement system to measure one or more mud properties of a mud being pumped into a well, a continuous drilling measurement system to measure one or more drilling parameters of the well being drilled, a data collection system connected to the mud measurement system and the drilling measurement system configured to synchronously collect the measured one or more mud properties and measured one or more drilling parameters, and a data analysis system connected in real time to the data collection system.

### BACKGROUND

To recover oil and gas from subsurface formations, wellbores (also referred to as boreholes) are drilled by rotating a drill bit attached at an end of a drill string. The drill string includes a drill pipe or a coiled tubing (referred herein as the “tubing”) that has a drill bit at its downhole end and a bottomhole assembly (BHA) above the drill bit. The wellbore is drilled by rotating the drill bit by rotating the tubing and/or by a mud motor disposed in the BHA. A drilling fluid, commonly referred to as the “mud” is supplied under pressure from a surface source into the tubing during drilling of the wellbore. The drilling fluid operates the mud motor (when used) and discharges at the drill bit bottom. The drilling fluid then returns to the surface via the annular space (annulus) between the drill string and the wellbore wall or inside. Fluid returning to the surface carries the rock bits (cuttings) produced by the drill bit as it disintegrates the rock to drill the wellbore.

In overburdened wellbores (when the drilling fluid column pressure is greater than the formation pressure), some of the drilling fluid penetrates into the formation, thereby causing a loss in the drilling fluid and forming an invaded zone around the wellbore. It is desirable to reduce the fluid loss into the formation because it makes it more difficult to measure the properties of the virgin formation, which are required to determine the presence and retrievability of the trapped hydrocarbons. In underbalanced drilling, the fluid column pressure is less than the formation pressure, which causes the formation fluid to enter into the wellbore. This invasion may reduce the effectiveness of the drilling fluid.

A substantial proportion of the current drilling activity involves directional boreholes (deviated and horizontal boreholes) and/or deeper boreholes to recover greater amounts of hydrocarbons from the subsurface formations and also to recover previously unrecoverable hydrocarbons. Drilling of such boreholes require the drilling fluid to have complex physical and chemical characteristics. The drilling fluid is made up of a base such as water or synthetic material and may contain a number of additives depending upon the specific application. A major component in the success the drilling operation is the performance of the drilling fluid, especially for drilling deeper wellbores, horizontal wellbores and wellbores in hostile environments (high temperature and pressure). These environments require the drilling fluid to excel in many performance categories. The drilling operator and the mud engineer determine the type of the drilling fluid most suitable for the particular drilling operations and then utilize various additives to obtain the desired performance characteristics such as viscosity, density, gelation or thixotropic properties, mechanical stability, chemical stability, lubricating characteristics, ability to carry cuttings to the surface during drilling, ability to hold in suspension such cuttings when fluid circulation is stopped, environmental harmony, non-corrosive effect on the drilling components, provision of adequate hydrostatic pressure and cooling and lubricating impact on the drill bit and BHA components.

A stable borehole is generally a result of a chemical and/or mechanical balance of the drilling fluid. With respect to the mechanical stability, the hydrostatic pressure exerted by the drilling fluid in overburdened wells is normally designed to exceed the formation pressures. This is generally controlled by controlling the fluid density at the surface. To determine the fluid density during drilling, the operators take into account prior knowledge, the behavior of rock under stress, and their related deformation characteristics, formation dip, fluid velocity, type of the formation being drilled, etc.

It is common to determine certain physical properties in the laboratories from fluid samples taken from the returning wellbore fluid. Such properties typically include fluid compressibility, rheology, viscosity, clarity and solid contents. The returning wellbore fluid is analyzed at the surface to determine the various desired chemical properties of the drilling fluid.

As noted above, a function of the drilling fluid is to transport cuttings from the wellbore as the drilling progresses. Once the drill bit has created a drill cutting, it should be removed from under the bit. If the cutting remains under the bit, it is re-drilled into smaller pieces, adversely affecting the rate of penetration, bit life and mud properties. The annular velocity needs to be greater than the slip velocity for cuttings to move uphole. The size, shape and weight of the cuttings determine the viscosity necessary to control the rate of settling through the drilling fluid. Low shear rate viscosity controls the carrying capacity of the drilling fluid. The density of the suspending fluid has an associated buoyancy effect on cuttings. An increase in density usually has an associated favorable effect on the carrying capacity of the drilling fluid. In horizontal wellbores, heavier cuttings can settle on the bottom side of the wellbore if the fluid properties and fluid speed are not adequate. Cuttings can also accumulate in washed-out zones.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments of the present disclosure relate to a system includes a continuous mud measurement system to measure one or more mud properties of a mud being pumped into a well, a continuous drilling measurement system to measure one or more drilling parameters of the well being drilled, a data collection system connected to the mud measurement system and the drilling measurement system configured to synchronously collect the measured one or more mud properties and measured one or more drilling parameters, and a data analysis system connected in real time to the data collection system.

In another aspect, embodiments of the present disclosure relate to a method that includes continuously measuring one or more mud properties of a mud being pumped into a well, continuously measuring one or more drilling parameters of the well being drilled, synchronizing the measured mud properties and the measured drilling properties to produce synchronized measured properties, and analyzing the synchronized measured properties.

### BRIEF DESCRIPTION OF DRAWINGS

FIG. 1 illustrates a schematic diagram of a drilling system, according to an embodiment.

FIG. 2 illustrates a flow diagram for a method of analyzing drilling parameters according to an embodiment.

FIG. 3 illustrates a flow diagram for analysis methods according to embodiments.

It should be noted that some details of the figures have been simplified and are drawn to facilitate understanding of the embodiments rather than to maintain strict structural accuracy, detail and scale.

### DETAILED DESCRIPTION

Reference will now be made in detail to embodiments of the present disclosure, examples of which are illustrated in the accompanying drawings. In the drawings and the following description, like reference numerals are used to designate like elements, where convenient. It will be appreciated that the following description is not intended to exhaustively show all examples, but is merely exemplary.

Embodiments of the present disclosure may provide drilling systems for determining various real time properties of the wellbore fluid during the drilling operations, including one or more of temperature and pressures, fluid density, accumulation of cuttings, viscosity, color, presence of methane and hydrogen sulfide, pH of the fluid, fluid clarity, and fluid flow rate. Such fluid properties, when taken in real-time, and synchronized with measured drilling parameters may be used to inform an operator about the drilling operation as it progresses. Parameters from the measurements may be computed by a processor at the surface. A surface computer or control system displays necessary information for use by the driller and may be programmed to automatically take certain actions, activate alarms if certain unsafe conditions are detected.

In the oil and gas industry, various devices and sensors have been conventionally been used to determine a variety of drilling parameters during drilling of wellbore, which include measurements made downhole or at the surface. Tools measuring the downhole properties are generally referred to as measurement-while-drilling (MWD) tools. The general emphasis of the industry has been to use MWD tools to determine parameters relating to the formations and the borehole, and the downhole tools in some instances. Measurements from the surface primarily may primarily involve parameters related to the drilling operation, such as torque, rate of penetration, etc. Conventionally, very few measurements are made relating to the drilling fluid. The majority of the measurements relating to the drilling fluid are made periodically at the surface, such as by analyzing samples collected from the fluid returning to the surface. While corrective actions may be taken based on such measurements, in many cases, such measurements take a long time and do not represent the actual fluid properties downhole. In contrast, embodiments of the present disclosure, by measuring/determining the fluid properties in real-time, the fluid may be directed related to the drilling parameters and well events, and the fluid properties may inform decision making analysis.

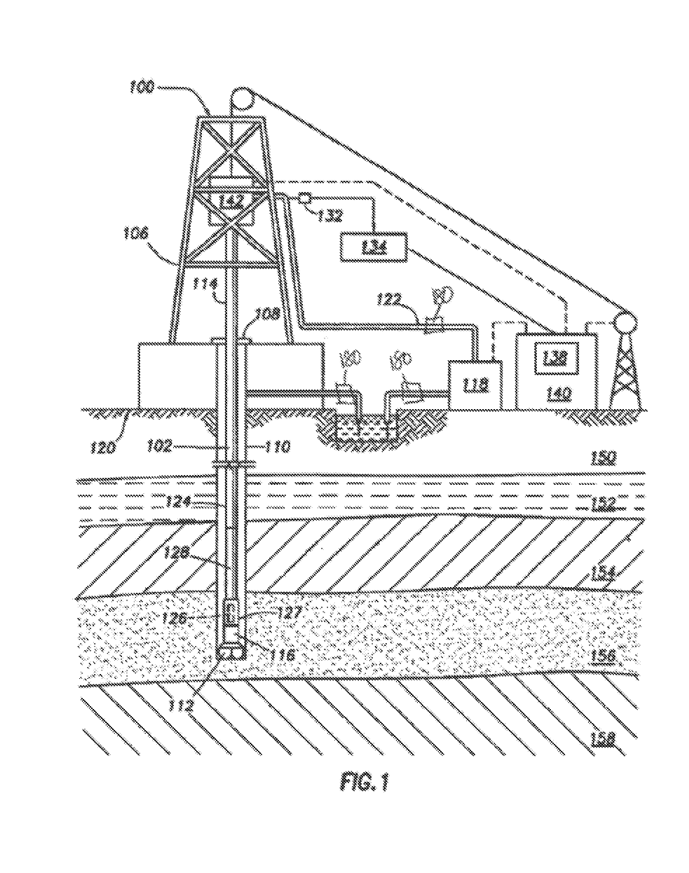

FIG. 1 illustrates a drilling system 100 that is equipped for real time data analysis of drilling and mud parameters. As shown in FIG. 1, the drilling system 100 includes a drill string 102 hanging from a derrick 106. A drill bit 112 is attached to the end of the drill string 102, and drilling is accomplished either by rotating via the top drive 142 and allowing the weight of the drill string 102 to press down on the drill bit 112 or by a rotary table 108 and kelly 114. The drill bit 112 may also be rotated independent of the drill string 102 by operating a downhole mud motor 116 above the drill bit 112.

While drilling, mud may be pumped from mud pumps 118 on the surface 120 through a standpipe 122 and down the drill string 102. The mud in the drill string 102 is forced out through jet nozzles (not shown) in the face of the drill bit 112 and returned to the surface through the well annulus 124, i.e., the space between the well 110 and the drill string 102. One or more sensors or transducers 126 may optionally be located in one or more measurement modules 127 in the bottomhole assembly of the drill string 102 to measure desired downhole conditions. Additional sensors may be provided as necessary to measure other drilling and formation parameters such as those previously described.

The measurements made by the transducers 126 may be transmitted to the surface, for example, through the drilling mud in the drill string 102, the mud pump 118, or any location on the surface. The transducers 126 send signals that are representative of the measured downhole condition to a downhole electronics unit 128. The signals from the transducers 126 may be digitized in an analog-to-digital converter. The downhole electronics unit 128 collects the binary digits, or bits, from the measurements from the transducers 126 and arranges them into data frames. Extra bits for synchronization and error detection and correction may be added to the data frames. The signal is transmitted according to known techniques, such as by carrier waveform through the mud in the drill string 102. The various electronics associated with mud pulse telemetry is known and for clarity is not further described. A pressure transducer 132 on the standpipe 122 detects changes in mud pressure and generates signals that are representative of these changes. The output of the pressure transducer 132 is digitized in an analog-to-digital converter and processed by a signal processor 134 which recovers the symbols from the received waveform and then sends the data to a computer 138. Other methods of downhole communication may be employed such as data transmission via wired drill-pipe.

Further, measurements are not only made downhole. Rather, embodiments of the present disclosure may relate to measurements that are made on the surface, from sensors on various pieces of rig equipment, including, but not limited to measurements used to calculate drilling parameters. For example, a hookload may be measured by hookload sensor mounted on deadline (not shown). Surface torque may be measured by a sensor device on the rotary table 108. Weight on the drill string/bit may be measured, for example, from one of several locations, including top drive 142 as well as the crown block (not shown, via a cable tension) at the top of derrick 106, or traveling block (not shown, adjacent top drive 142). Block position and the related block velocity may be measured by a block sensor on the traveling block or part of the draw works 182. Surface torque may be measured by a sensor device on the rotary table 108. At top drive 142, torque and RPMs may also be measured. Distance measurements from the traveling block may indicate the total depth drilled as well as rate of penetration. In some embodiments, a surface equipment control system 140 is configured to communicate with and control the operation of the various machinery at the well-site. For example, the surface equipment control system 140 transmits control signals and receives feedback from the top drive 142 to adjust and maintain drillstring rpm, the mud pump 118 to adjust the flow of drilling mud through the system and the winch drive (not shown) to adjust and maintain weight-on-bit. The surface equipment control system may be configured to communicate and control many other surface machinery which affects downhole operations. In one or more embodiments, the drilling parameter data measured may include data related to the drilling dynamics generally includes weight on bit (WOB), rate of penetration (ROP), torque on bit, vibration, bit type, bit diameter, caliper, and downhole temperature and pressure. Measurements of such drilling parameters may be made continuously during the drilling process. As used herein, continuously or continuous measurement refers to the frequent and regular measurement of the drilling parameters or properties, for example at a preselected drilling depth interval (such as 10 ft or less of drilling depth) or within a preselected time interval (such as every 10, 5, or 1 minutes or even less). Further, it is also understood that the interval may be changed during the drilling process, particularly as the well reaches greater depths, risks, etc., and/or that the continuous measurement may be turned on or off for certain sections of the wellbore being drilled.

In one or more embodiments, one or more sensors or transducers 180 may continuously measure fluid properties and convey the data to the computer 138. As mentioned above, continuously or continuous measurement refers to the frequent and regular measurement of the properties, for example at a preselected drilling depth interval (such as 10 ft or less of drilling depth) or within a preselected time interval (such as every 10, 5, or 1 minutes or even less). As described in greater detail below, in accordance with embodiments of the present disclosure, the continuous measurement of the drilling parameters and the continuous measurement of the fluid properties may be synchronized. As used herein, synchronized (as well variations of the word, such as synchronous or synchronously) refer to measurements or data collection that occur at the same time and/or with reference to a common or master clock. By synchronizing the two types of data, the data may be analyzed in the manner that allows for statistical correlations or conclusions to be drawn about how the fluid properties and drilling parameter effects the well being drilled (as well as for the drilling to be adjusted accordingly).

The types of fluid property measurements may include the mud being pumped into the well (at the surface) but may also include measurement of the mud as it is circulated out of the well. Examples of properties for which measurements may be taken, include, but are not limited to the following (ranges of data are included in parentheses): a) temperature (5 to +200° C.); b) mud weight/density (500-3000 g/l); c) oil/water (0-100%); d) viscosity (10-150000 Cp or 10-500000 Cp); e) rheology (10-500000 Cp at 3, 6, 30, 60, 100, 200, 300 and 600 rpm); f) gel strength (at 10 seconds, 10 minutes and 30 minutes); h) pH (0-14); i) hardness/conductivity (5 μS/cm-2000 mS/cm); j) solids and liquids breakdown (0-100%); k) sand content/particle size analysis (7 micron-48 mm); l) pressure (100 mbar-40 bar); m) API fluid loss; n) filter cake; o) calcium, magnesium, chloride, potassium, lime, and clay content; p) HTHP filter; q) electric stability, and r) pump rate.

In some embodiments, the one or more sensors or transducers 180 may be located in a conduit for suction to the mud pumps or a parallel branch line, or connected to mud pits, and/or also in the mud return flow downstream or upstream or both downstream and upstream of the shale shakers. In some embodiments, the transducers 180 may also be arranged upstream of the mud pump, for measuring mud property parameters as delivered and pumped down the well. In some embodiments, the transducers 180 are located in the mud pump suction line. The transducers 180 may have a pressure rating that is compatible with the mud pressure from the mud pump if installed on the delivery side of said pump. The transducers 180 deliver real time data from the overall mud system or for specific data, directly to a user or the computer 138 from the transducers and processing software. Thus, transducers may measure fluid properties before and/or after the fluid is circulated downhole. It is also envisioned that the fluid properties may involve a measurement of the amount of cuttings removed from a fluid that returns to the surface after being circulated downhole. Specifically, the amount of cuttings collected from one or more shakers or other separation devices may be measured and provide an indication of the amount of rock removed from the well (and thus, how well the hole is cleaned).

The one or more sensors or transducers 180 may be of the type certified to operate in gaseous hazardous atmospheres, arranged in an optimized skid, with a certified hazardous atmosphere electrical, optical or wireless connection to a control room or similar having analysis and data storage capacity feasible for the mud engineer, or having real time connection to the mud engineer. In some embodiments, the analysis and data storage capacity may also be available to others located at the drill site, for example, the driller's chair, or at remote locations. Feasible instrumentation is commercially available in the market, from the shelf or on order from producers or suppliers.

Further, in addition to the above described measurements, it is also envisioned that the present method and system may use different types of data collected at the rig site which may include both sensor and non-sensor data, and/or which may include data collected from different types of sources, such as from different types of sensors, from video, from cameras, from personnel manually inputting data, and/or from automatic data input (e.g., computer inputted commands to control one or more components may be automatically collected). Sensor data may be used to refer to data collected from one or more sensors disposed along a well construction system. Non-sensor data may be used to refer to data collected from ways other than through sensors. For example, non-sensor data may include data collected manually by personnel operating or servicing one or more components in the well construction system. According to embodiments of the present disclosure, non-sensor data may include, for example, at least one of operational state data (e.g., data indicating an on/off status of one or more components in the well construction system), operator commands, observational inputs, and interactions between personnel and the well construction system.

Sensor data may be collected via electrical signals transmitted from the sensor through a data transmission system to a computing device, a database, a data bus, or a network. A data transmission system may include wires in communication between sensor(s) and a computing device and/or may include wireless transmission of signals from sensor(s) to a remote receiver.

According to embodiments of the present disclosure, sensor data may include data collected from one or more sensors related to an operation process, such as standpipe pressure, flow rate, hookload, block position, and others, including those described above. Sensor data may further include data collected from one or more sensors related to a condition of one or more components in a well system as well as the fluid properties.

Another type of collected data may include non-sensor data collected from operator commands, for example, an operator command to set a selected RPM (revolution per minute) set point (e.g., set point for rotational speed of drill pipe or rotational speed of a motor), an operator command to set a selected weight-on-bit (WOB) set point, an operator command to set a pump flow rate (e.g., through stroke count), or other operator commands to control an equipment unit. Data collected from operator commands may be captured through an automated control system (e.g., a computer-inputted command to control one or more components may be transmitted and stored in a database storing collected data), or through manual input (e.g., an operator may manually input a command made to a well construction system into a database storing collected data).

Collected data may also include data related to personnel interaction with the well construction system. Data collected from personnel interaction with a well construction system may include different types of information about what personnel do to the well construction system, such as material preparation (e.g., adding a selected amount of barite into a circulating subsystem of the well construction system or preparing a number of barrels of lost circulation material) and maintenance information (e.g., changing a shaker screen, replacing a mud pump liner, etc.). Data collected from personnel interaction with a well construction system may be captured through an active sensing system (e.g., with one or more sensors, through video, etc.), or through manual input (non-sensor data). For example, non-sensor data collected from interactions between personnel and a well construction system may include, for example, manual input of a description of measurements taken and/or servicing performed such as replacing a component, etc.

Another type of collected data may include observational inputs, which includes information describing what personnel observe about a well construction system. For example, observational inputs may include data inputted describing observation of the operational state of an equipment unit (e.g., if a mud pump breaks down, if operation delay is due to a particular reason) or data inputted describing one or more conditions of a component (e.g., if a pipe fitting is leaking). Further, observational inputs may be captured through personnel description of their visual observation, or through a camera or video.

Another type of collected data may include data corresponding to the operating state of an equipment unit, which may be collected from one or more sensors (sensor data) and/or without the use of a sensor (non-sensor data). For example, data associated with equipment and its operating state may be captured through a direct sensor measurement, such as a valve position sensor, or through indirect measurement, such as current to a mud pump. Data associated with equipment and its operating state may also be captured through manual input of an observation, such as noting an equipment unit out of service. Equipment operating state data may include, for example, data indicating if the equipment is on, off, open, closed, and maintenance information (e.g., if the equipment is undergoing maintenance, the last date/time the equipment was maintained, the type of maintenance conducted, if a component was replaced, and other maintenance data).

Collected data may also include data that relates to general monitoring. For example, as mentioned above, collected data may come from video. Collected data may also come from audio devices and/or vibration sensors (attached to a structure of a well construction system), which may be recorded and archived along appropriate time-stamps. Video, audio and vibrational data can be processed using predetermined algorithms to generate certain state variables, for example, the number and location of people in an office or on a rig floor. According to some embodiments, a computer program executing a video analyzing algorithm may be used to detect and identify changes in collected video data, where the operational state of the at least one component may be determined based on the identified changes in the video.

Additionally, in combination with other data, learning algorithms can associate particular sensor behavior with different rig activities. Once the association has been learnt, the learnt association may allow additional redundancy to compensate if more specific sensors fail, and/or to detect sensor failures. For example, when a top drive rotation generates a vibration signal at the rotation speed of the top drive motor under normal operating conditions, if the top drive is operational but there is no vibration signal at the right frequency, a failure in the top drive rotation speed sensor may be indicated.

Collected data may be acquired automatically and/or manually. Manually collected data may be collected from manual inputs (e.g., from an operator and/or other personnel) of data into a computing device, and may include, for example, data describing observations of the well construction system and/or interactions with the well construction system entered by the personnel making the observation or interaction. For example, a person may manually input observed data through a computing device and include location information (e.g., referencing one or more components listed in the digital description of the well construction system) associated with the inputted data. Automatically collected data may be captured through instrumentation (e.g., sensors, cameras, video, etc.), where location information may be automatically tagged to associate the collected data with the corresponding location in the digital description of the well construction system. Automatically collected and manually collected data may be captured with a proper timestamp (e.g., corresponding to a master clock) indicating the time of data capture.

As data (non-sensor data and sensor data) is collected, the data may be time stamped from a single time reference, which may be referred to as a master clock reference. According to embodiments of the present disclosure, the systems and methods of the present disclosure may include a single time reference, which may be used to tag data collected throughout the entire well system. In this manner, multiple types of data collected from through the well system may be synchronized with a single master clock reference, which may then be used for the time stamping of the data as it is collected. Thus, use of such master clock may allow for the fluid properties data to be analyzed in combination with the drilling parameters data so that a drilling process may be optimized on the basis of a statistical analysis involving both fluid data and drilling parameters that correspond to the measured fluid data.

Further, it is also envisioned that the methods may include tagging collected data with an identification indicating at least one of a location of a source of collected data and a component in the well construction system from which the data is collected. For example, a location of a source of collected data may be defined in terms of its relationship to one or more components of the well construction system, e.g., a location of collected flow rate data from a sensor may be referenced in terms of the pipe on which the flow rate sensor is disposed. In some embodiments, sensor data may be acquired through an Electronic Data Recorder (EDR), where location information in reference to the well construction system and a timestamp may be captured as meta data associated with the sensor data. Using the location information and the digital description of the well construction system, the exact location of the placement of the sensors on the well construction system may be identified.

As mentioned above, in one or more embodiments, the various measurements may be synchronized with a single master clock reference so that the drilling parameter data received and analyzed by the computer 138 is analyzed with reference to the corresponding fluid data. In some embodiments, the computer 138 may include an interface that will display the properties measured. The real time interface to the mud engineer or driller's chair may comprise a display visualizing the measured mud properties. The computer 138 is also connected to a database including mud properties and well property data, empirical and theoretical, and the computer 138 includes real time connection to the instrumentation arranged operatively to the mud flow and drilling parameters. The computer 138 may include or is coupled to statistical analysis algorithms, for using real time quality data of properties of mud to be pumped down the well and/or real time quality data on mud return flow properties analyzed in combination with the measured drilling parameters. The mud data may include comparison of pumped in mud properties with returned mud properties, for generating estimates and proposals for future action. The statistical analysis may also compare the measured drilling parameters so that, based on the mud data and the observed drilling data, a statistical analysis may be performed for generating estimates and proposals for future action. In some embodiments, the computer 138 may be located at the drilling rig or located at a remote location.

The drilling parameters and the mud measurements may be used together, optionally with other real time measurements, i.e., those received from downhole tools, including any other data being measured/collected for the drilling process. The collected data from the mud measurements and drilling parameters, which may be synchronized onto a common clock, may allow for a side-by-side comparison of the fluid is being or was pumped downhole and the drilling parameters as the well is drilled. This synchronization may be used to assess the cause or reason for an event occurrence or may be used to predict (and/or avoid) future events.

Further, it also may allow for optimization of a drilling process in a manner that has not been performed heretofor. Generally, the drilling process is selected in a manner with a single focus: how to drill faster. However, in accordance with the present disclosure, the drilling process optimization may have a multi-variate statistical analysis to achieve a more economical approach. For example, the general approach of how to drill faster focuses the analysis on a single variable: the daily cost of the rig—using the presumption that drilling faster reduces the number of days of drilling and thus the number of days the rig is rented. However, in accordance with the present disclosure, in addition to the cost of the rig, the analysis may also consider the cost of the fluid(s) needed to finish drilling and complete the well, the cost of the tools associated with such drilling plan, the cost of cleaning the well. Thus, the methods and systems of the present disclosure consider that while drilling faster may reduce the ultimate rental cost of the rig (to drill to total depth), it may reduce the hole cleaning (if the cuttings are unable to be brought out of the well by the fluid having its given properties), and thus necessitate further actions to adequately clean the well and prepare it for completions. Thus, one or more embodiments of the present disclosure may use a multi-variate statistical analysis that optimizes the drilling process in order to obtain a more economical plan. For example, the statistical analysis may conclude that a more economical plan may be achieved by slowing down (reducing the ROP), pumping faster, pumping a more viscous fluid, substituting a fluid chemistry (for example, to a cheaper fluid and slowing down or to more expensive fluid and speeding up).

The collection and storage of drilling fluid and drilling parameter data on a single master clock for a given well or wells may allow for such stored data to for a data repository that may subsequently be used on a neighboring or separate well as the basis of a statistical analysis. Such statistical analysis may be performed without consideration or involvement of models that correlate two parameters (such as torque and weight-on-bit) together. While it may be generally understood (based on theory or empirical observation) that such parameters are related, there is no integral model taking all factors into consideration. However, the present disclosure uses a statistical approach which allows for the drilling fluid properties to be statistically correlated with observed drilling parameters, where the two are taken on or synchronized to a single reference clock. Embodiments may use historical statistical correlation and/or contemporaneous data to provide a statistical correlation between the real-time drilling fluid and drilling parameter data.

Embodiments provide methods for using surface measurements (e.g., data acquired wholly or substantially from data which may be collected from measurements made at the surface) for drilling parameter optimization and analysis, including proactive and post-event analysis.

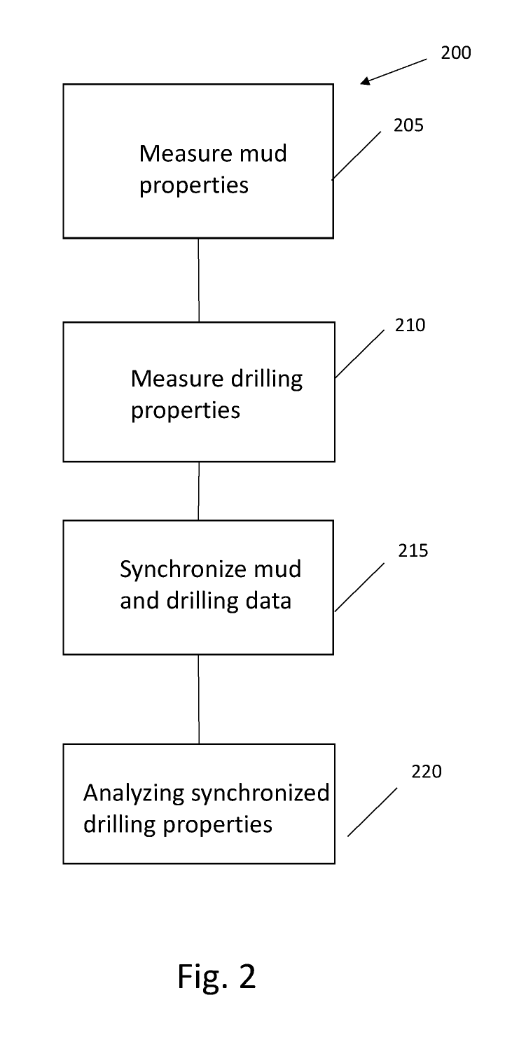

FIG. 2 illustrates a flow diagram for a method 200 of analyzing the above described real time collected mud properties and drilling parameters according to some embodiments. The method 200 includes continuously measuring mud properties in step 205. As described above the mud properties may be collected via the one or more sensors and transducers 180 and sent to the computer 138. The mud properties are measured in real time or in frequent time increments as described above. Thus, as used herein, continuous measurement refers to the frequent and regular measurement of properties (mud or drilling) for example at a preselected drilling depth interval (such as 10 ft or less of drilling depth) or within a preselected time interval (such as every 1, 5, or 10 minutes). Further, it is also understood that the interval may be changed during the drilling process, particularly as the well reaches greater depths, risks, etc.

The method also includes continuously measuring drilling properties in step 210. As described above the drilling properties may be collected via the one or more sensors and transducers 126 and sent to the computer 138. The drilling properties are measured in real time or in frequent time increments as described above.

The measured mud properties and the measured drilling properties may be synchronized in step 215. Synchronizing the measured mud properties and the measured drilling properties includes placing the data onto a common clock. Synchronization of the data may allow for the mud properties and the drilling parameters to be considered in combination together on a continuous (and in real-time) basis. Thus, the synchronized drilling properties are analyzed in step 220. The synchronization of the two types of data and analysis of the synchronized data may allow an operator to understand, as the well is being drilled, i.e., in real-time, what drilling parameters result from a particular mud that is being pumped downhole. The mud that is being pumped downhole, particularly as it is returned to the surface, cleaned, and recirculated, may have varying properties, and the continuous measurement of mud properties, synchronized with the drilling parameters may allow an operator to correlate observed drilling parameters or events with the mud properties, and thus make real-time corrections or adjustments to the drilling as it proceeds. For example, the adjustments that may be made may include slowing down the drilling speed (reducing the ROP), pumping at different rate, pumping a different viscosity fluid, substituting a fluid chemistry. These changes to the drilling process may be made in real-time, i.e., the data may be measured/collected/synchronized/analyzed at the rig site and the drilling may be changed based on the synchronized data almost instantaneously (recognizing that some changes to fluid system, etc., may take more time than, for example, changing the fluid flow rate).

In some embodiments, other data from the drilling process which has been measured and sent to the computer 138, as described above, may also be placed on the common clock. In other embodiments, the measured mud properties and the measured drilling properties may be synchronized on a common network and is done simultaneously when the measuring of the mud properties and the measuring of the drilling properties occurs.

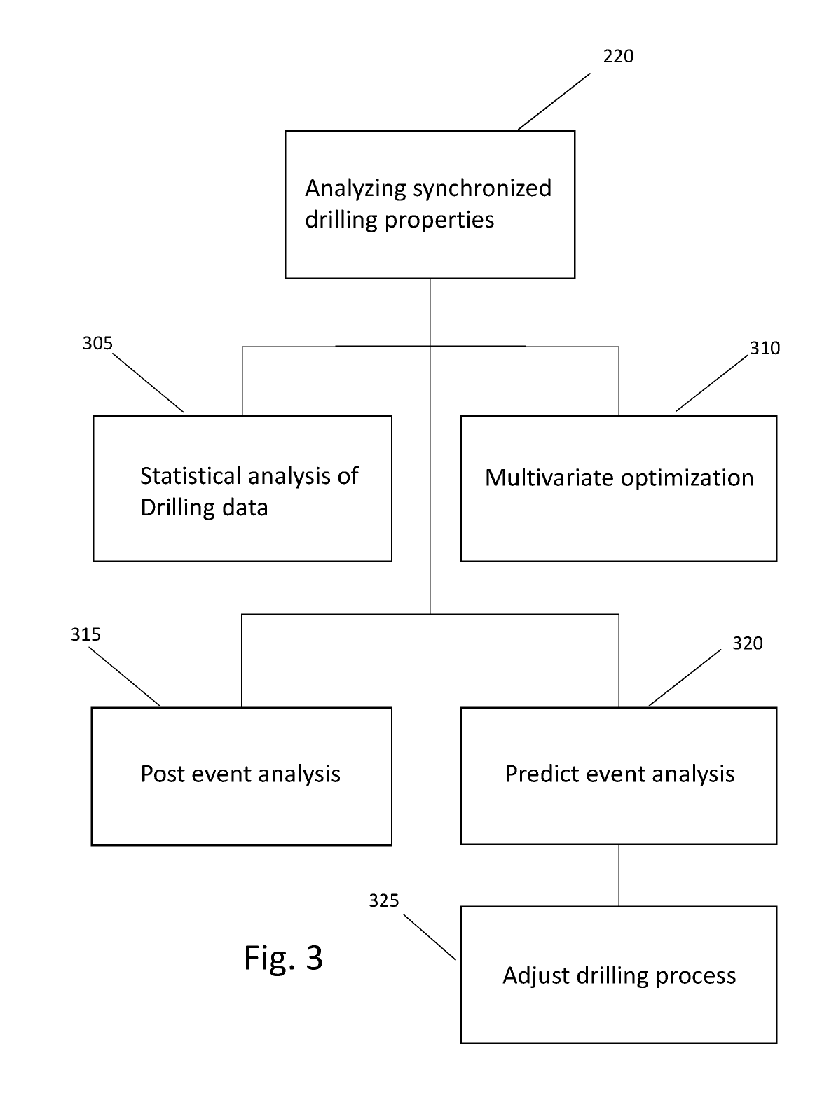

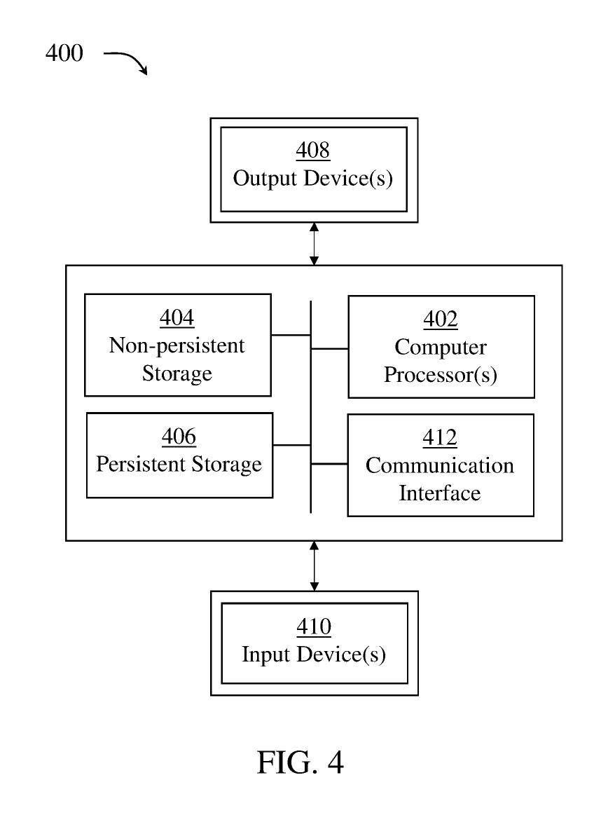

FIG. 3 illustrates a flow diagram for a variety of ways the synchronized data may be analyzed. The synchronized mud and drilling properties may provide large amounts of data that can be manipulated and analyzed in a variety of ways. The synchronized data may be statistically analyzed at 305, used to optimize the drilling operation at 310, for predictive analysis in step 315 and for post event analysis in step 320.

Generally, the analysis and drilling of oilwells has been controlled by the judgment and direct human actions of the driller operating the mechanical and electrical systems of the drilling rig. The driller will typically directly control at the surface control station, for example, drill pipe speed and position, the vertical force applied to drillstring, the rotary speed of the drillstring and the flowrate of the drilling fluid. These parameters, among others, may be controlled within limits such as the physical limitations of the rig equipment, or in some cases, pre-defined limits of the input or output parameter, e.g. the torque applied to the drillstring can be limited. The drillers choice of parameters is the result of his general understanding of the feedback responses he gets from the surface equipment, and general observation. This is imperfect information since it does not typically include direct information about the downhole behavior of the drillstring, the formations being drilled or to be drilled, and their relation to the input parameters at surface and the resulting consequences and efficiencies.

Existing analysis of the drilling operations provided to/by a drill operator, in many cases, restrict maximum efficiency, at least due to the fact that the calculations are merely forecasts of the expected drilling properties and earth formations. For this reason, the operations limits, typically provided in absolute parameter values such as an actual rpm, are heavily diluted with error margins. Further, the limits have been developed to generically apply to the entire depth of a borehole, and are not dependent on the specific formation properties encountered.

Approaches have been attempted to refine the limits based on substantial changes to the drilling process. However, even this effort is typically left to human initiative. Thus, to the extent operating guidelines can be modified during the drilling process, substantial risks of human error are introduced into sensitive drilling operations. For this reason, most modifications to drilling processes have been left to the experience of the drilling operator. However, a drill operator's capability to perform certain analyses is limited both by time (limited time to perform testing and calculations) and human ability (limited to relatively simple comparisons). Further, even when a manual analysis is made, the process of implementing a modification introduces error in part due to the drill operator matching to absolute parameter values, many times using analog instrumentation. These limitations in turn introduce inconsistent drilling practices as new drilling operators rotate across work shifts.

To assist in minimizing drilling operation inconsistency, charts have been developed which provide points of reference for some of the drilling parameters. For example, a chart may list a range of drill rpms and a range of downward bit weights to determine an adequate mud flow rate. However, these charts, like the original drilling operations limits, are calculated well in advance of the actual drilling and are thus based on predictions of the drilling conditions. Further, a basic limitation of the charts is due to the inherent finite restriction of the discrete data points, requiring the operator to interpolate between the available data points to fit the actual conditions in order to deduce the proper drilling modification. Thus, by analyzing the synchronized fluid properties and drilling parameters, a driller may guide the drilling process using such data instead of or in addition to intuition. Further, use of the synchronized data may allow for trends, risks, etc. to be identified in a much shorter period of time and/or with a much greater sensitivity than what is feasibly based on manual observation and intuition. For example, a small change in the fluid viscosity may be undetected by an operator, and when coupled with the given flow rate, may result in insufficient hole cleaning, that can only be detected over a larger period of time. Similarly, a small change in the fluid viscosity may be undetected by an operator, and when coupled with the given flow rate, may result in a larger impact than what would be expected by the operator, and thus the operator may be making assumptions without understanding the changes in the fluid viscosity. The operator, having the benefit of a synchronized data (of mud and drilling parameters) may be able to make adjustments to the drilling process sooner or more appropriately than what is feasible without such synchronized data.

To perform the statistical analysis at 305, the statistical analysis may find trends and correlations based on the synchronized data. Further, the multivariate optimization at 310 may take into account the different costs of operating the drilling operation, the risks associated with the drilling operation. The drilling operations may be modeled and refined by finding relationships between the measured mud properties and the measured drilling properties along the common timeline. In some embodiments, the costs may include rig time, fluids, tools and cleaning/tripping. The proposed optimization may be used in the decision making by the mud engineer to modify the mud parameters and/or drilling parameters. Other data may be included in developing the correlations. For example, core or seismic data may be included as inputs in the correlation development phase, and rock hardness or brittleness may be predicted properties. In order to identify correlations between inputs such as drilling dynamics data and mud log data, and outputs such as logs and core analysis, the processor may employ neural networks and/or genetic algorithms to determine the correlations, although other algorithms known in the art may be used.

Further, also in the optimization, the synchronized data in the computer may be used to minimize the risks of the drilling operation. In other embodiments, the synchronized data may be used to modify the drilling operation to change the mud properties in the drilling operation or to change the drilling parameters. The optimization may provide the most financially efficient drilling operation. In some embodiments, the driller's decision may be based on information from the drilling operation. The driller's decision may include, but is not limited to, slowing down the drill, pumping faster, changing the composition of the fluids or any combination thereof.

In some embodiments, to provide the most financially efficient drilling operation, the costs of the drilling operation are included in the optimization step 310. Costs associated with, but not limited to, the drilling operation include the rig (time), fluids, tools and cleaning/tripping. These costs may be analyzed to provide optimal drilling operations, i.e., balancing the costs versus the risks. In some embodiments, the decision making may include changing the drilling operation (e.g. pumping faster or slowing drill speed) or changing the composition of the drilling fluid. The decision making may be moved from the drilling engineer to the computer, which may use historical data and statistical analysis to optimize the drilling operation.

The synchronized data may be used for predictive analysis in step 320. Predictive analysis may encompass using the synchronized data and in some embodiments, historical data, to predict potentially negative events and potentially provide an alarm or warning to the drilling rig. It is understood that such predictive analysis may occur during the drilling of the well, in real-time, so that the drilling underway may be adjusted at step 325. By analyzing correlations between the measurements performed at the surface of the first wellbore and the drilling parameters, trends and correlations may be found that can be used for further refining the model or to optimize the drilling operation. In some embodiments, the predictive analysis may be done to slow down the drilling prior to an incident occurring. In some embodiments, a decision tree analysis may be determined based on the associated risks. The decision tree analysis may be used to make decisions based on outcomes determined from model and/or decision tree.

The synchronized data may also be used for post event analysis in step 315. Post event analysis may encompass using the synchronized data and in some embodiments, historical data, to analyze an event and review the actions taken. This will help and further refine the model or to determine changes to the drilling parameters to avoid events. The analysis may also be used to predict events and to refine the model. Specifically, the statistical analysis of the synchronized data described herein may have a greater accuracy as the model builds a larger data set on which the statistical analysis may be based. Thus, embodiments of the present disclosure may also relate to the synchronous measurement and analysis of mud properties and drilling parameters for application in subsequent wells.

Embodiments may be implemented on a computing system. Any combination of mobile, desktop, server, router, switch, embedded device, or other types of hardware may be used. For example, as shown in FIG. 4, a computing system 400 may include one or more computer processors 402, non-persistent storage 404 (e.g., volatile memory, such as random access memory (RAM), cache memory), persistent storage 406 (e.g., a hard disk, an optical drive such as a compact disk (CD) drive or digital versatile disk (DVD) drive, a flash memory, etc.), a communication interface 412 (e.g., Bluetooth interface, infrared interface, network interface, optical interface, etc.), and numerous other elements and functionalities.

The computer processor(s) 402 may be an integrated circuit for processing instructions. For example, the computer processor(s) may be one or more cores or micro-cores of a processor. The computing system 400 may also include one or more input devices 410, such as a touchscreen, keyboard, mouse, microphone, touchpad, electronic pen, or any other type of input device.

The communication interface 412 may include an integrated circuit for connecting the computing system 400 to a network (not shown) (e.g., a local area network (LAN), a wide area network (WAN) such as the Internet, mobile network, or any other type of network) and/or to another device, such as another computing device.

Further, the computing system 400 may include one or more output devices 408, such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s) 402, non-persistent storage 404, and persistent storage 406. Many different types of computing systems exist, and the aforementioned input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the disclosure. The computing system 500 in FIG. 5 may be connected to or be a part of a network. In one or more exemplary embodiments, the functions described may be implemented in hardware, software, firmware, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

While the present teachings have been illustrated with respect to one or more embodiments, alterations and/or modifications may be made to the illustrated examples without departing from the spirit and scope of the appended claims. In addition, while a particular feature of the present teachings may have been disclosed with respect to only one of several implementations, such feature may be combined with one or more other features of the other implementations as may be desired and advantageous for any given or particular function. Furthermore, to the extent that the terms “including,” “includes,” “having,” “has,” “with,” or variants thereof are used in either the detailed description and the claims, such terms are intended to be inclusive in a manner similar to the term “comprising.” Further, in the discussion and claims herein, the term “about” indicates that the value listed may be somewhat altered, as long as the alteration does not result in nonconformance of the process or structure to the illustrated embodiment. Finally, “exemplary” indicates the description is used as an example, rather than implying that it is an ideal.

Other embodiments of the present teachings will be apparent to those skilled in the art from consideration of the specification and practice of the present teachings disclosed herein. It is intended that the specification and examples be considered as exemplary only, with a true scope and spirit of the present teachings being indicated by the following claims.

What is claimed is:

1. A system comprising:
a continuous mud measurement system to measure one or more mud properties of a mud being pumped into a well;
a continuous drilling measurement system to measure one or more drilling parameters of the well being drilled;
a data collection system connected to the mud measurement system and the drilling measurement system configured to synchronously collect the measured one or more mud properties and measured one or more drilling parameters; and
a data analysis system connected to the data collection system.

2. The system of claim 1, wherein the data analysis system comprises a multi-variate economic analysis.

3. The system of claim 1, wherein the mud measurement system comprises one or more mud measurement sensors arranged in a suction line to one or more mud pumps, in a mud pit, or in a mud return flow conduit.

4. The system of claim 3, wherein the one or more mud measurement sensors comprise a Coriolis meter, an infrared instrument, an x-ray fluorescence instrument, a viscosity meter or rheology sensor.

5. The system of claim 1, wherein the drilling measurement system comprises one or more drilling measurement sensors located at the surface of the well.

6. The system of claim 1, wherein the mud measurement system and the drilling measurement system are on a common master tune clock.

7. The system of claim 1, wherein the data analysis system comprises statistical analysis to provide correlations and trends based on the collected data.

8. A method comprising:
continuously measuring one or more mud properties of a mud being pumped into a well;
continuously measuring one or more drilling parameters of the well being drilled;
synchronizing the measured mud properties and the measured drilling properties to produce synchronized measured properties; and
analyzing the synchronized measured properties.

9. The method of claim 8, further comprising adjusting one or more mud properties and/or one or more drilling parameters in the well being drilled based on the analyzed synchronized measured properties

10. The method of claim 8, wherein the analysis comprises a multivariate economic analysis and the method further comprises optimizing a drilling plan based on the multivariate economic analysis.

11. The method of claim 10, further comprising drilling the well according to the optimized drilling plan.

12. The method of claim 10, wherein the optimized drilling plan comprises changing one or more of a fluid composition, a fluid viscosity, a fluid flow rate, or a rate of penetration.

13. The method of claim 10, wherein the analyzing comprises predicting optimized mud properties for a well to be drilled.

14. The method of claim 10, wherein the analyzing comprises predictive event analysis.

15. The method of claim 14, further comprising triggering a warning signal based on the predictive event analysis.

16. The method of claim 10, wherein the analyzing comprises post event analysis.

17. The method of claim 10, wherein the analyzing comprises determining a cost efficient drilling process.

18. The method of claim 17, wherein the cost efficient drilling process comprises cost of rig, cost of fluids, cost of tools, cost of cleaning/tripping

19. The method of claim 8, wherein the one or more mud properties are selected from the group consisting of temperature, mud weight or density, Oil/water ratio, viscosity, rheology, gel strength, pH, hardness/conductivity, solids and liquids breakdown, sand content, particle size analysis, pressure, API fluid loss, filter cake, calcium content, magnesium content, chloride content, potassium content, lime content, clay content, HTHP filter, electric stability, or pump rate.

20. The method of claim 8, wherein the one or more drilling parameters are selected from the group consisting of torque on bit, weight on bit, rate of penetration (ROP), and drill string RPM.

21. The method of claim 10, further comprising
measuring one or more return mud properties of a mud being returned from a well.

22. The method of claim 20, wherein the one or more return mud properties are selected from the group consisting of density, viscosity, flow rate, pressure, temperature, presence of gas (methane, CO2 and H2S), fluid density, viscosity, pH, solid content, fluid clarity, fluid compressibility, fluid rheology and spectral analysis.
